(19) United States
(12) Patent Application Publication
(10) Pub. No.: US 2020/0046774 A1
(43) Pub. Date: Feb. 13, 2020

(54) COMPOSITION FOR PREVENTING OR TREATING BONE DISEASES WHICH HAS EXCELLENT BONE REGENERATION EFFECT

(71) Applicant: KOREA UNIVERSITY RESEARCH AND BUSINESS FOUNDATION, Seongbuk-gu Seoul (KR)

(72) Inventors: Jae Jun SONG, Seocho-gu Seoul (KR); Ho Seok SONG, Gwacheon-si Gyeonggi-do (KR); Yoon Young GO, Guro-gu Seoul (KR)

(21) Appl. No.: 16/486,757

(22) PCT Filed: Feb. 14, 2018
(86) PCT No.: PCT/KR2018/001910

§ 371 Date: Oct. 23, 2019

(30) Foreign Application Priority Data
Feb. 16, 2017 (KR) 10-2017-0020967
Feb. 12, 2018 (KR) 10-2018-0017137

(51) Int. Cl. A61K 35/28; A61K 35/32; A61K 35/33; A61K 35/545

(52) U.S. Cl. CPC A61K 35/28; A61K 35/545; A61K 35/33; A61K 35/32

(57) ABSTRACT

The present invention relates to a composition for preventing or treating bone diseases, comprising a BMP-2-encoding gene and HSV-tk-encoding gene, and as an active ingredient a stem cell into which a dual kill switch expression vector in which an HGPRT-encoded gene is knocked out is introduced or a cell differentiated from the stem cell, wherein the bone regeneration effect is realized by a BMP-2 growth factor and at the same time, apoptosis may also be dually controlled by the dual kill switch.

### TECHNICAL FIELD

Example embodiments of the present disclosure relate to a composition for preventing or treating bone diseases which has an excellent bone regeneration effect, and includes, as an active ingredient, a stem cell with a double safety mechanism to realize a bone regeneration effect using a BMP-2 growth factor and at the same time to control apoptosis using a dual kill switch.

### BACKGROUND ART

Bones that support human body's soft tissues and body weight and protect internal organs from external shocks, are one of important parts of a human body which not only structurally support muscles or organs but also store calcium or other essential minerals, such as phosphorus or magnesium, in the body.

Bones of a fully-grown adult have been balanced by continually repeating a generation and absorption process of removing old bones and replacing the old bones with new bones, which is called “bone remodeling” (Yamaguchi A. et al., Tanpakushitsu Kakusan Koso., 50 (6Supp1); 664-669, 2005). Such bone circulation is essential for restoring fine bone damage caused by growth and stress and for maintaining a function thereof. In adults, about 10% to 30% of skeleton is reshaped every year through remodeling of bone resorption-bone formation.

Osteoblasts that generate bones and osteoclasts that break down bones are involved in bone remodeling, and bone homeostasis is maintained through a close interaction between osteoblasts and osteoclasts. For example, osteoblasts may maintain bone homeostasis in a body by regulating differentiation of osteoclasts that function for bone resorption through secretion of materials such as a receptor activator of nuclear factor-κB ligand (RANKL) and osteoprotegerin (OPG) that is an induction receptor thereof.

In the past, research has been conducted mainly on metabolic abnormalities of bone minerals, that is, calcium and phosphorus, in connection with treatment of bone diseases accompanying when a bone tissue is damaged or when homeostasis of bones is not maintained due to physical effects, hormone systems, and the like, and no progress has been made in identifying a mechanism thereof.

Generally, a diet containing calcium is recommended for treatment and prevention of osteoporosis, and administration of estrogen or vitamin D is recommended for menopausal women. In addition, bisphosphonate-based drugs such as Fosamax® (component name: alendronate) and Actonel® (component name: risedronate) are attracting attention as new alternative therapeutic agent as bone resorption inhibitors that inhibit osteoclasts and induce death.

However, although a calcium adjuvant widely used as a bone disease therapeutic agent inhibits secretion of parathyroid hormone and prevents a decrease in a bone mass due to bone resorption, it is known that an individual difference in maintenance of the bone mass is severe (Heandy R. P. principles of bone biology, Academic press, 1007-1017, 1996). In addition, hormone therapy using estrogen or calcitonin has been reported to increase a bone density and reduce incidence of rectal cancer, but side effects such as breast cancer, myocardial infarction and venous thrombosis have been reported (Nelson, H. D et al., JAMA, 288: 872-881, 2002; Lemay, A., J. Obstet. Bynaecol. Can., 24: 711-7152-3).

Recently, the number of cases in which necrosis of a jawbone, severe atrial fibrillation, incapacitation of bones or joints, or musculoskeletal pain occurs in patients taking bisphosphonate preparations is increasing every year (Coleman R E., Br J Cancer, 98: 1736-1740 (2008). Thus, there is a need for development of a new bone disease therapeutic agent that may effectively suppress bone resorption while having fewer side effects.

After a fracture, various growth factors allow a damage site and distant mesenchymal stem cells to become mature osteocytes through recruitment/differentiation, and bone regeneration occurs through generation of a new blood vessel and a bone tissue.

Here, BMP-2 that is a protein that performs a key function in recruitment and differentiation of stem cells is known as a growth factor that plays the most crucial role in bone regeneration. A BMP-2 growth factor in the form of a recombinant protein is used as a growth factor in the form of injections in clinical trials because a bone regeneration effect is recognized. However, since more than 80% of BMP-2 growth factors are released when BMP-2 growth factors are initially injected, BMP-2 growth factors are not efficiently used.

Existing methods for bone regeneration treatment include a surgical treatment, a treatment using biomaterials/tissue engineering, and a treatment using stem cells/growth factors. In autogenous bone grafting in bone grafting, a donor site may be injured, and a repetitive surgery may be required. In allogeneic bone/heterogeneous bone grafting, bone regeneration may lack, or complications such as infections may occur.

A biomaterial scaffold has a disadvantage in that stem cells or growth factors are required due to a weak bone regeneration effect in the case of great bone defects caused by a lack of osteoinduction. Currently, a bone regeneration treatment using stem cells/growth factors is developed at a minimal level, and thus there is a need to develop a functional cell therapeutic agent that may enhance a bone regeneration ability through combination of stem cells and growth factors.

Since a cell therapeutic agent may be differentiated into unwanted cells within a body and inhibit a function of a tissue, or may develop into a malignant tumor, safety issues thereof also need to be solved.

### DISCLOSURE OF INVENTION

### Technical Goals

The present disclosure is to solve the foregoing problems, and an aspect of the present disclosure is to provide a composition for preventing or treating bone diseases which has an excellent biosafety and enhances a bone regeneration effect.

However, the problems to be solved in the present disclosure are not limited to the foregoing problems, and other problems not mentioned herein would be clearly understood by one of ordinary skill in the art from the following description.

### Technical Solutions

According to an aspect of the present disclosure, there is provided a composition for preventing or treating a bone disease, including a BMP-2-encoding gene and an HSV-tk-encoding gene, and a stem cell into which a dual kill switch expression vector in which an HGPRT-encoding gene is knocked out is introduced, or a cell differentiated from the stem cell, as an active ingredient.

The stem cell may be an embryonic stem cell (ESC) or a mesenchymal stem cell (MSC).

The cell differentiated from the stem cell may be a fibroblast or an osteoblast.

The fibroblast may be a teratoma-derived fibroblast (TDF).

The bone disease may be at least one selected from the group consisting of a bone defect, osteoporosis, an osteoporotic fracture, a diabetic fracture, a nonunion fracture, osteogenesis imperfecta and osteomalacia.

The composition may increase expression of at least one marker selected from the group consisting of alkaline phosphatase (ALP), integrin binding sialoprotein (IBSP), runt-related transcription factor 2 (RUNX2), osterix (OSX), secreted phosphoprotein 1 (SPP1) and osteocalcin (OCN).

The composition may further include a scaffold.

The scaffold may include polycaprolactone (PCL) or biphasic calcium phosphate (BCP).

According to another aspect of the present disclosure, there is provided a method of preparing a composition for preventing or treating a bone disease, including preparing a vector including a BMP-2-encoding gene and an HSV-tk-encoding gene; knocking out an HGPRT-encoding gene in the vector; and introducing the vector into a stem cell or a cell differentiated from the stem cell.

### Effects

A composition for preventing or treating bone diseases according to the present disclosure may include a stem cell into which a BMP-2 gene is introduced, and thus it is possible to generate a BMP-2 growing factor using the stem cell, thereby realizing an excellent bone regeneration effect.

Also, the stem cell may include an HSV-tk gene, and a dual kill switch may be implemented by knocking out an HGPRT-encoding gene, and thus it is possible to dually prevent cells from becoming cancer cells due to self-proliferation, and the like, and possible to enhance human safety.

The effects of the present disclosure are not limited to the above-described effects, and it should be understood that the effects include all effects that can be inferred from the configuration of the invention described in the detailed description of the present disclosure or the appended claims.

### BRIEF DESCRIPTION OF DRAWINGS

FIG. 1 illustrates BMP-2 and HSV-tk gene expression and cell morphologies before and after a Cre treatment according to an example embodiment.

FIG. 2 is a graph illustrating a BMP-2 release behavior of a cell line according to an example embodiment.

FIG. 3 is a graph illustrating an ALP activity of a cell line according to an example embodiment.

FIG. 4 is a graph illustrating an ALP activity of a TDF cell line into which a BMP-2 is injected from the outside according to an example embodiment.

FIG. 5 is a graph illustrating a BMP-2 release behavior in osteogenic differentiation of a cell line according to an example embodiment.

FIG. 6 is a graph illustrating a gene expression level associated with osteogenic differentiation of a cell line according to an example embodiment.

FIG. 7 is a graph illustrating a calcium deposition level of a cell line according to an example embodiment.

FIG. 8 is a graph illustrating a mineral deposition level of a cell line according to an example embodiment.

FIG. 9 illustrates X-ray images showing osteogenesis effects of a femoral defect animal model of cell lines according to an example embodiment.

FIG. 10 illustrates micro-CT results showing osteogenesis effects of cell lines according to an example embodiment.

FIG. 11 illustrates H&E tissue staining results of cell lines showing osteogenesis effects of a cranial defect animal model, and a graph for a comparison of a degree of decrease in a size of a defect region according to an example embodiment.

FIG. 12 illustrates images showing cell morphologies based on a gancyclovir treatment concentration and time in cell lines according to an example embodiment.

FIG. 13 is a graph illustrating a number of cells based on a gancyclovir treatment concentration and time.

FIG. 14 illustrates images obtained by capturing cell line morphologies according to an example embodiment.

FIG. 15 is a graph illustrating BMP-2 release behaviors of cell lines according to an example embodiment.

FIG. 16 is a graph illustrating a gene expression level associated with osteogenic differentiation of a cell line according to an example embodiment.

FIG. 17 is a graph illustrating a calcium deposition level of a cell line according to an example embodiment.

FIG. 18 is a graph illustrating a mineral deposition level of a cell line according to an example embodiment.

FIG. 19 illustrates H&E tissue staining results of cell lines showing osteogenesis effects of a cranial defect animal model according to an example embodiment.

FIG. 20 illustrates images showing resistance test results for 6-TG of cell lines according to an example embodiment.

FIG. 21 illustrates images showing apoptosis effects of aminopterin on cell lines according to an example embodiment.

FIG. 22 illustrates images showing a bone regeneration marker expression level of a cell line according to an example embodiment.

FIG. 23 is a graph illustrating a comparison of a BMP-2 release behavior based on whether lgK is attached.

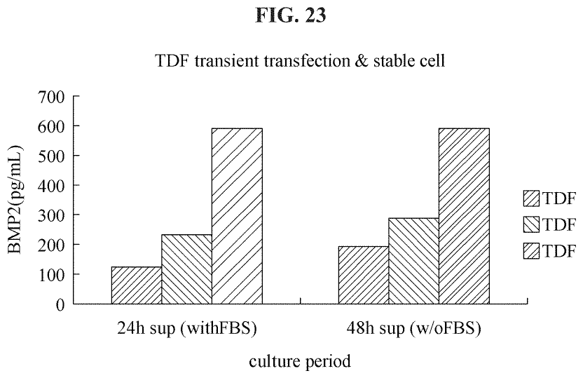

FIG. 24 is a graph illustrating a comparison of an HGPRT expression level between a control group and a dual kill switch group after HGPRT gene editing.

### BEST MODE FOR CARRYING OUT THE INVENTION

Hereinafter, example embodiments will be described in detail with reference to the accompanying drawings. Like reference numerals illustrated in the drawings refer to like constituent elements throughout the specification.

Various modifications may be made to example embodiments. However, it should be understood that these example embodiments are not construed as limited to the illustrated forms and include all changes, equivalents or alternatives within the idea and the technical scope of this disclosure.

The terminology used herein is for the purpose of describing particular example embodiments only and is not intended to be limiting of the invention. As used herein, the singular forms “a,” “an” and “the” are intended to include the plural forms as well, unless the context clearly indicates otherwise. It will be further understood that the terms “include” and/or “have,” when used in this specification, specify the presence of stated features, integers, steps, operations, elements, components or combinations thereof, but do not preclude the presence or addition of one or more other features, integers, steps, operations, elements, components, and/or groups thereof

Unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When describing the example embodiments with reference to the accompanying drawings, like reference numerals refer to like constituent elements and a repeated description related thereto will be omitted. When it is determined detailed description related to a related known function or configuration which may make the purpose of the present disclosure unnecessarily ambiguous in describing the present disclosure, the detailed description will be omitted here.

According to an example embodiment, there is provided a composition for preventing or treating a bone disease. The composition may include a BMP-2-encoding gene and an HSV-tk-encoding gene, and a stem cell into which a dual kill switch expression vector in which an HGPRT-encoding gene is knocked out is introduced, or a cell differentiated from the stem cell, as an active ingredient.

The BMP-2 is a type of bone morphogenetic proteins that are involved in healing of cartilage-resistant membrane fractures, that promote a bone growth and that are necessary for natural regeneration reactions, and may enhance a bone disease treatment effect by introducing the BMP-2 to be directly produced in cells in comparison to when BMP-2 is injected from the outside.

Hypoxanthine-guanine phosphoribosyltransferase (HGPRT) refers to a protein that inhibits apoptosis, and removing an HGPRT-encoding gene may be a double safety mechanism to prevent a failure in control of apoptosis due to a loss of HSV-tk. In other words, by inserting an HSV-tk encoding gene into an expression vector, a single kill switch may be implemented, and a dual kill switch may also be implemented by knocking out the HGPRT-encoding gene. For example, when a dual kill switch expression vector is introduced in a cell line, apoptosis may be induced by treatment of drugs such as aminopterin. Thus, the composition according to the present disclosure may effectively control apoptosis, to prevent side effects of stem cell therapeutic agents, for example, an abnormal growth, creation of malignant tumors, and the like.

A cell line into which the BMP-2 is introduced may be a stem cell, and the stem cell may be an embryonic stem cell (ESC) or a mesenchymal stem cell (MSC). The stem cell may perform an osteogenesis function thereof separately from bone formation by the BMP-2, but may realize a more effective osteogenesis effect in comparison to when the stem cell is applied together with the BMP-2.

Also, the cell differentiated from the stem cell may be a fibroblast or an osteoblast. The fibroblast may be a teratoma-derived fibroblast (TDF).

In other words, a cell into which the BMP-2-encoding gene is introduced may be a TDF formed from an ESC, and an osteoblast differentiated from the ESC, but is not limited thereto.

The term “teratoma” used herein is a type of tumors formed of various cells and tissues such as skin cells, muscle cells, and nerve cells, unlike a general tumor formed of a single cell, and may be formed by injecting the ESC into a mouse. Fibroblasts generated from the teratoma may be isolated and used as cell lines, and thus an expression efficiency of BMP-2 may be enhanced. Also, the fibroblasts may be differentiated into osteoblasts, and thus an osteogenesis function may be implemented.

Due to an excellent differentiation ability of the stem cell or the cell differentiated from the stem cell, the stem cell or the cell differentiated from the stem cell may simultaneously have a risk of developing into cancer cells as well as a function as a therapeutic agent.

Thus, a BMP-2 gene together with a suicide gene, such as an HSV-tk gene, may be introduced into the cell line, thereby preventing the cell line from being developed into cancer cells.

An internal ribosome entry site (IRES) gene may be inserted between the BMP-2 gene and HSV-tk gene. By using the IRES gene, both the BMP-2 gene and HSV-tk gene may be expressed as a single promotor. The BMP-2 gene, the IRES gene, and the HSV-tk gene may have base sequences represented by SEQ ID NO: 1, SEQ ID NO: 2, and SEQ ID NO: 3, respectively.

Also, the BMP-2 gene may be obtained by introducing a gene that encodes a Lgk peptide at a 5′ end. The Lgk peptide may be a leader peptide that induces extracellular release of intracellularly produced BMP-2, and may enhance a bone formation effect by the BMP-2. A BMP-2 gene into which a Lgk peptide-encoding gene is introduced may have a base sequence represented by SEQ ID NO: 4.

The HSV-tk gene may phosphorylate drugs such as acyclovir and gancyclovir, and phosphorylation of the drugs may induce apoptosis by inhibiting a DNA synthesis by DNA polymerase. Thus, cells of the cell line may be prevented from becoming cancer cells by administering drugs such as acyclovir and gancyclovir at a point in time at which bone formation by BMP-2 is sufficiently induced in the cell line. In other words, the above HSV-tk gene may act as a kill switch for the cell line.

The BMP-2-encoding gene and the HSV-tk-encoding gene may be inserted into a single expression vector, and may be expressed by transfection of the expression vector into a host cell. The expression vector may include, for example, but is not limited to, an adenovirus expression vector, an adeno-associated virus vector, a retroviral vector, or plasmids.

The bone disease to be prevented or treated by the composition may be a disease associated with a decrease in a bone mass or a bone density, and may be, but is not limited to, at least one selected from the group consisting of a bone defect, osteoporosis, an osteoporotic fracture, a diabetic fracture, a nonunion fracture, osteogenesis imperfecta and osteomalacia.

Thus, the composition may increase expression of a gene or a protein associated with an increase in a bone mass or a bone density. For example, the composition may increase expression of at least one marker selected from the group consisting of alkaline phosphatase (ALP), integrin binding sialoprotein (IBSP), runt-related transcription factor 2 (RUNX2), osterix (OSX), secreted phosphoprotein 1 (SPP1) and osteocalcin (OCN), however, there is no limitation thereto.

Although the composition itself exhibits an excellent osteogenic induction ability, the composition may further include a scaffold to further enhance the effect. The scaffold may be formed of, but is not limited to, polycaprolactone (PCL) or biphasic calcium phosphate (BCP). The scaffold may perform a function of fixing a cell line included in the composition at an implanted position.

According to an example embodiment of the present disclosure, there is provided a method of preparing a composition for preventing or treating a bone disease, including preparing a vector including a BMP-2-encoding gene and an HSV-tk-encoding gene; and introducing the vector into a stem cell or a cell differentiated from the stem cell.

A function of each of the BMP-2-encoding gene and the HSV-tk-encoding gene, a method of introducing the BMP-2-encoding gene and the HSV-tk-encoding gene into a cell line, and a type of bone diseases are the same as those described above. A method of knocking out the HGPRT-encoding gene may use a known method of removing a gene, and may be performed using a TAL-nuclease, a meganuclease, a zinc-finger nuclease (ZFN), or an RNA-guided endonuclease. According to an example embodiment, a Cas9/CRISPR method may be used.

Hereinafter, the present disclosure will be described in more detail with reference to examples. The following examples are given for the purpose of illustrating the present disclosure, and the scope of the present disclosure is not limited thereto.

### EXAMPLE 1

### Establishment of TDF Cell Line

10⁶ WA01 male embryonic stem cells (WiCell research institute) were mixed with 30% Matrigel (BD biosciences), and injected subcutaneously into severe combined immunodeficiency (SCID) mice. After 6 weeks, formed teratomas were isolated, pulverized to 1 mm³, and then cultured in a DMEM culture solution containing 10% FBS, 1% nonessential amino acid, 1% penicillin-steptomycin, 2 mM glutamax and 55 μM β-mercaptoethanol. During the culturing, teratoma-derived fibroblasts (TDFs) grown from a teratoma tissue were isolated by trypsin, and subcultured, to establish a cell line.

### EXAMPLE 2

### Preparation of Vector into Which BMP-2-Encoding Gene and HSV-tk-Encoding Gene are Inserted

A pL453 vector containing a CAG promoter-loxP-neo-loxP was cut with a NotI restriction enzyme, and blunt ends were formed using T4 DNA polymerase. Plasmid DNA containing an HSV-tk gene was cut with a BglII/NcoI restriction enzyme, blunt ends were formed using T4 DNA polymerase, and inserted into the vector.

Next, the vector was cut with a BamHI restriction enzyme, blunt ends were formed using T4 DNA polymerase, and then a BMP-IRES portion was amplified by a PCR and inserted into the vector, to prepare a vector into which a BMP-2-encoding gene and an HSV-tk-encoding gene were inserted.

### EXAMPLE 3

### Introduction of Vector into Cell Line

The TDF cell line of Example 1 was dispensed in a 60-phi dish to obtain 2×10⁶ cells, and then the vector of Example 2 was transfected. After 48 hours had elapsed after transfection, cells that do not contain a neoR gene were killed and cells into which the BMP-2 gene was introduced were selected, through treatment with neomycin for 5 days.

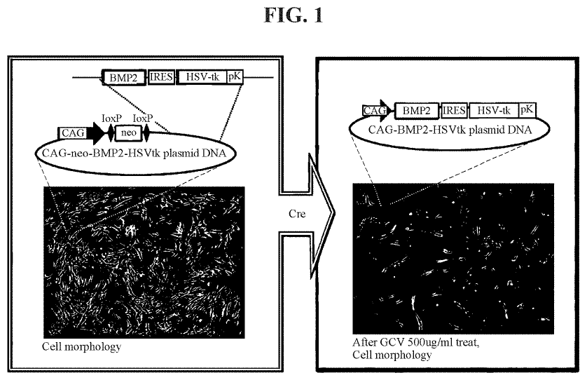

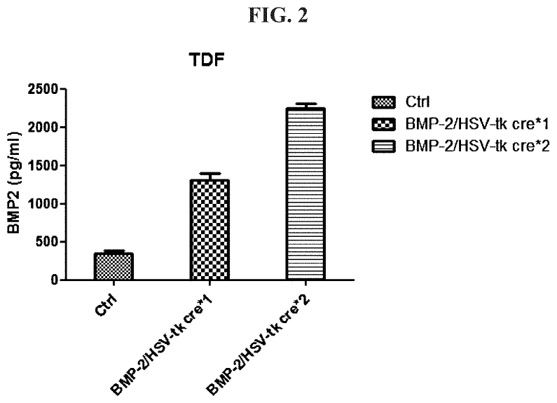

Next, plasmid DNA containing pCAG-Cre was transfected into the cell, and a loxP gene and Cre protein were reacted, to allow expression of BMP-2 and HSV-tk genes (FIG. 1). Expression of BMP-2 was confirmed by an ELISA analysis, and the results are shown in FIG. 2. Referring to FIG. 2, when treatment with Cre was performed twice, extracellular release of about 2 ng/ml of BMP-2 is confirmed.

### EXAMPLE 4

### Evaluation of Early Osteogenic Induction Ability

To evaluate an early in vitro osteogenic induction ability of a cell line according to Example 3, an activity level of alkaline phosphatase (ALP) that is an index of an early stage of osteogenesis was measured.

To measure an ALP activity of each of the TDF cell line of Example 3 and a normal TDF cell line in which BMP-2 is not expressed, a group in which cells were cultured in an osteogenesis induction medium (OIM), and a group in which cells were cultured in a general growth medium (GM) were separately cultured for 3 days and 7 days, and then each ALP activity was measured.

After 3 days and 7 days of the culturing, samples were washed twice with a PBS solution, and dissolved in a cell lysis buffer to which 0.1% Triton X-10 was added.

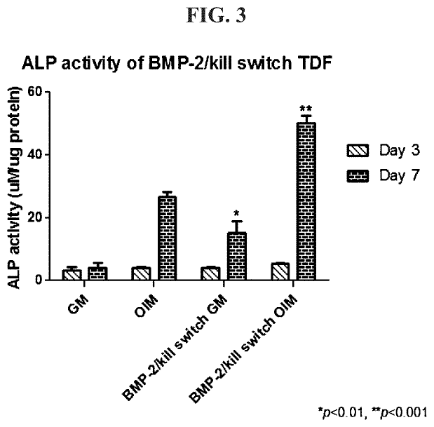

Lysed cells were centrifuged at 4° C. and 13000 rpm for 30 minutes, and then a supernatant was recovered and quantitated by a Bradford assay. The ALP activity was measured at 405 nm for the same amount of proteins using an ALP kit (AnaSpec), and the results are shown in FIG. 3.

Referring to FIG. 3, when 7 days had elapsed after induction of osteogenesis, the ALP activity of the cell line of Example 3 was observed to be about two times higher than that of the control group, and the cell line of Example 3 exhibited the ALP activity even in the general GM. Thus, it may be found that an excellent early osteogenic induction ability may be realized by a TDF cell line into which BMP-2 is introduced.

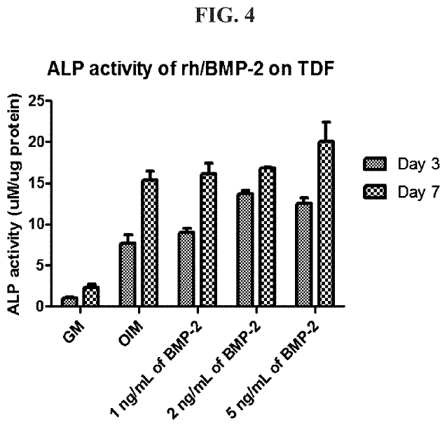

To evaluate an osteogenic induction ability when BMP-2 is injected from the outside in comparison to the TDF cell line of Example 3, a commercial recombinant BMP-2 protein was injected into a normal TDF cell line, the same experiment as that described above was performed, and the results are shown in FIG. 4.

Referring to FIG. 4, when 7 days had elapsed after induction of osteogenesis, a statistically significant ALP activity was not observed in all experimental groups in which treatment with 1 ng/ml, 2 ng/ml and 5 ng/ml of BMP-2 was performed in comparison to the control group that was not treated with BMP-2. It may be found that considering that a concentration of BMP-2 released from TDF cells of Example 3 is 2 ng/ml as described above, a TDF cell line that directly expresses BMP-2 therein may realize a significantly excellent osteogenic induction ability in comparison to when BMP-2 is injected from the outside.

### EXAMPLE 5

### Evaluation of BMP-2 Release Behavior During Osteogenesis

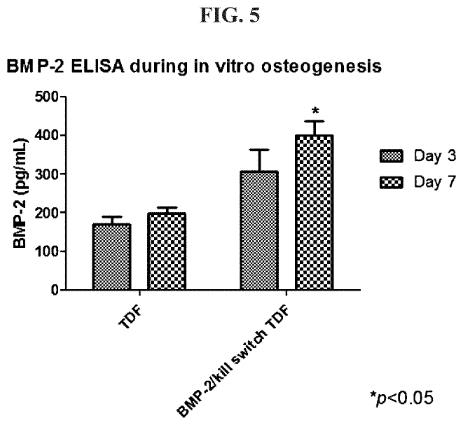

To verify whether the TDF cell line of Example 3 continues to maintain BMP-2 release during osteogenesis, a BMP-2 release behavior was analyzed through an ELISA analysis when 3 days and 7 days had elapsed after the TDF cell line of Example 3 was cultured in an OIM, and the results are shown in FIG. 5.

Referring to FIG. 5, it may be confirmed that BMP-2 release from the TDF cell line of Example 3 is enhanced in comparison to a TDF cell line that was not treated at points of time of both 3 days and 7 days after the culturing. Thus, it may be found that the TDF cell line of Example 3 may continue to maintain BMP-2 release during the osteogenesis.

### EXAMPLE 6

### Measurement of Osteogenesis-related Marker Expression Level

To verify whether the TDF cell line of Example 3 increases expression of alkaline phosphatase (ALP) and integrin binding sialoprotein (IBSP) shown in an early osteogenesis stage, and runt-related transcription factor 2 (RUNX2) and osterix (OSX) that are transcription factors related to osteogenic differentiation, gene expression levels of the above markers were measured by extracting RNA of all the TDF cell line of Example 3 and TDFs into which BMP-2 was not introduced using a TRIzol reagent (Invitrogen) when 3 days had elapsed after induction of osteogenic differentiation of the TDF cell line and the TDFs.

Each of the cultured cells was washed twice with PBS, lysed with 1 ml of the TRIzol reagent, 200 μl of chloroform was added thereto, stirred, and centrifuged at 4° C. and 12000 rpm for 20 minutes to separate a supernatant. 500 μl of isopropanol was added to the separated supernatant, stirred, and centrifuged again at 4° C. and 12000 rpm. A pellet, except the supernatant, was washed three times with 70% ethanol, and RNA was isolated.

5 μl of each RNA was prepared, added to an RT-PCR amplification kit, and reacted at 45° C. for 60 minutes, to construct cDNA. The constructed cDNA was amplified by a PCR using primers specific for ALP, osteocalcin (OCN) and osteopontin (OPN) in real time.

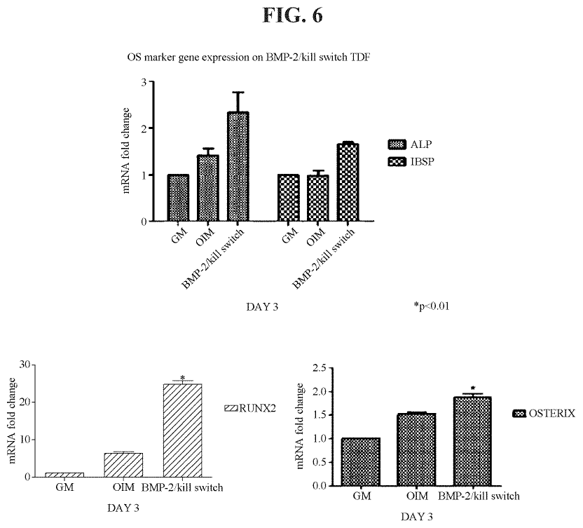

10 μl of 2× SYBR green reagent (Roche) and each primer (0.5 pmol/μl) were added by 1 to the same amount of cDNA, reacted 40 times at 95° C. for 30 seconds and at 60° C. for 1 minute and amplified, and RT-PCR results are shown in FIG. 6.

Referring to FIG. 6, it may be found that the TDF cell line of Example 3 increases gene expression levels of ALP, IBSP, RUXN2 and OSX that are osteogenic markers, which implies that a TDF cell line into which BMP-2 is introduced may effectively induce osteogenesis.

### EXAMPLE 7

### Evaluation of Late Osteogenic Induction Ability

To evaluate a late osteogenic induction ability of the TDF cell line of Example 3, each of the TDF cell line of Example 3 and a normal TDF cell line that does not express BMP-2 was cultured in an OIM or a general GM for 10 days to 12 days, and a calcium deposition level and a mineral deposition level were measured.

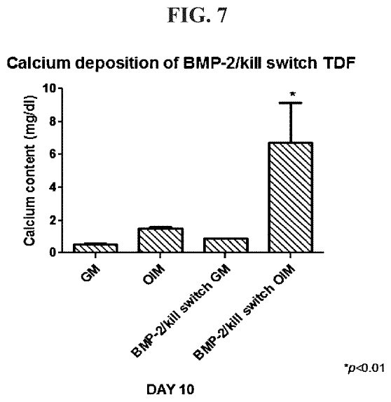

The calcium deposition level was measured using a QuantiChrom™ calcium assay kit (DICA-500). Samples were acquired when 10 days had elapsed after induction of osteogenic differentiation, washed twice with a PBS solution, treated with 0.6N HCl, and stored at 4° C. for 24 hours. Calcium deposited in cells was measured at 612 nm using the calcium assay kit, and the results are shown in FIG. 7.

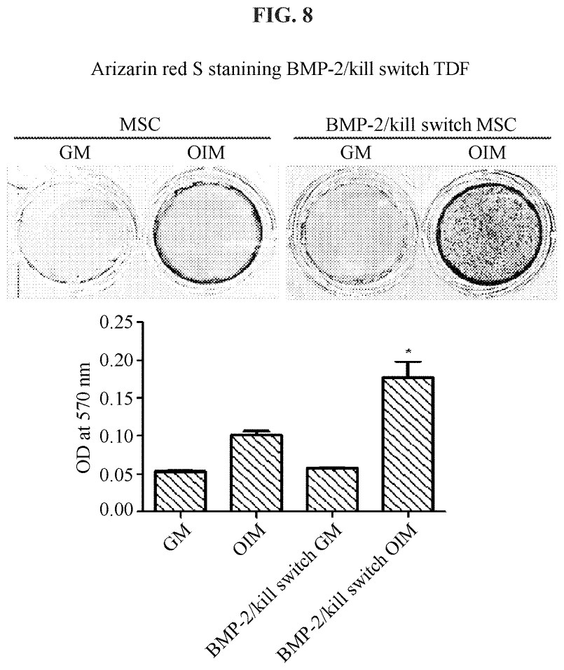

The mineral deposition level was measured using an arizarin red S staining solution (Millipore). After 10 days of induction of osteogenic differentiation, samples were washed twice with a PBS solution, and cells were fixed in 4% paraformaldehyde for 15 minutes. A fixing solution was removed, washed with distilled water, and the arizarin red S staining solution was added thereto, and stored at room temperature for 20 minutes. After staining was completed, the cells were washed three times with distilled water, to verify a color change and at the same time, to measure an optical density (OD) at 570 nm, and the results are shown in FIG. 8.

Referring to FIGS. 7 and 8, it may be found that the TDF cell line of Example 3 exhibits a calcium deposition level at least four times greater than the control group and a mineral deposition level at least twice greater than the control group, and accordingly the late osteogenic induction ability is excellent.

### EXAMPLE 8

### Evaluation of Osteogenesis in Animal Model

To verify a bone regeneration effect of the TDF cell line of Example 3 in a femoral defect animal model, a bone defect model was prepared by segmental resection of an ilium after surgically exposing thighs of 7-week-old rats. After 4 weeks and 8 weeks of the segmental resection, a bone defect of 7 mm in size was prepared to prevent union.

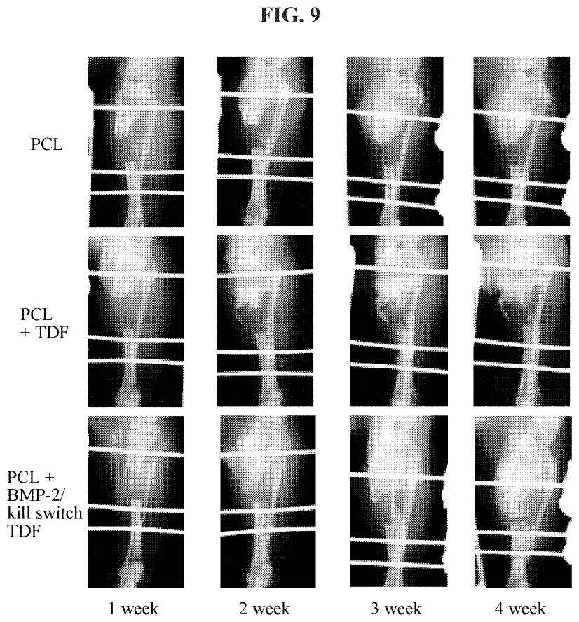

5×10⁵ TDFs of Example 3 and 5×10⁵ TDFs of the control group into which BMP-2 was not introduced were mixed with polycaprolactone (PCL) scaffolds, cultured at 37° C. for 24 hours, and then introduced into bone defect regions of the rats. A surgical site was sutured and fixed with an external fixator, and then bone formation was observed using X-ray at weekly intervals, and the results are shown in FIG. 9.

Referring to FIG. 9, it may be found with naked eyes that osteogenesis is increased in both a group in which the TDFs of the control group were injected and a group in which the TDFs of Example 3 were injected, in comparison to a group in which only PCL scaffolds were injected, after 2 weeks of bone defect. In particular, when 4 weeks had elapsed after the bone defect, union of the TDFs of Example 3 in the bone defect region is more effective than that of the TDFs of the control group.

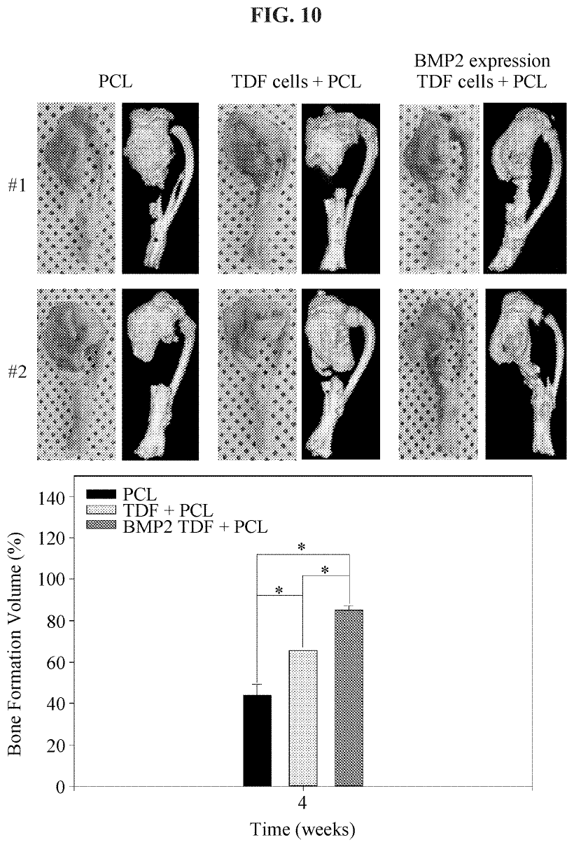

Also, after 4 weeks of the bone defect, ilium defect regions of the rats were captured by micro-CT, and bone formation sites were measured and compared, and the results are shown in FIG. 10. Referring to FIG. 10, it may be confirmed that the group in which the TDF cell line of Example 3 was introduced exhibits a significantly enhanced osteogenesis ability in comparison to the group in which only the PCL scaffolds were introduced and the control group in which a TDF cell line was introduced.

To verify a bone regeneration effect of the TDF cell line of Example 3 in a cranial defect animal model, a bone defect model was prepared by segmental resection after surgically exposing skulls of 8-week-old nude mice. After 4 weeks and 8 weeks of the segmental resection, a bone defect of 4 mm in size was prepared to prevent union.

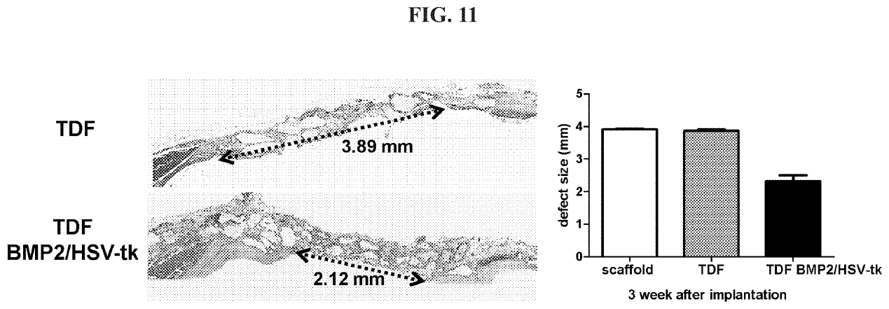

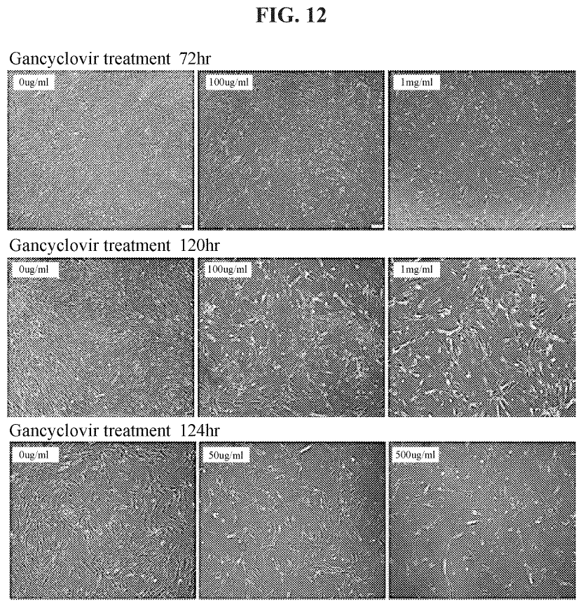

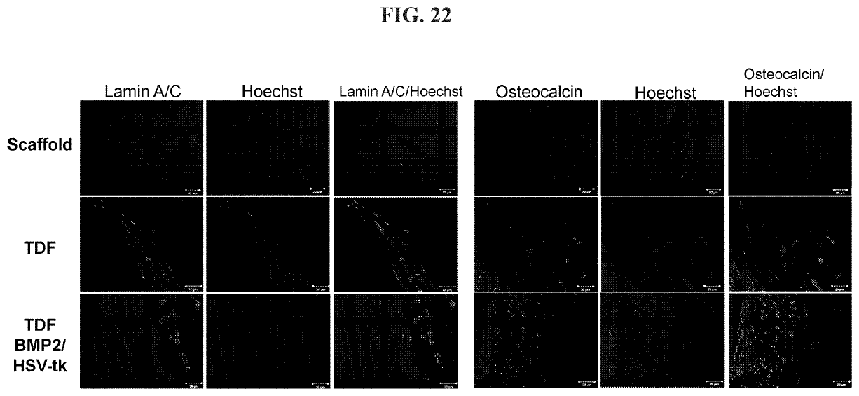

5×10⁵ TDFs of Example 3 and 5×10⁵ TDFs of the control group into which BMP-2 was not introduced were mixed with biphasic calcium phosphate (BCP) scaffolds, cultured at 37° C. for 24 hours, and then introduced into bone defect regions of the rats. After 3 weeks, cranial defect regions were collected, osteogenesis was evaluated using an H&E tissue staining test method, and the results are shown in FIG. 11. Results obtained by performing fluorescence staining of osteocalcin and Lamin A/C markers are shown in FIG. 12. Referring to FIG. 11, it may be confirmed that the group in which the TDF cell line of Example 3 was introduced exhibits an excellent osteogenesis effect in comparison to the control group in which a TDF cell line was introduced, and reduced a largest number of defect regions. Referring to FIG. 22, it may be confirmed that in the group in which the TDF cell line of Example 3 was introduced, osteocalcin and Lamin A/C that are bone regeneration markers are remarkably expressed in comparison to the control group in which the TDF cell line was introduced, which indicates that the group in which the TDF cell line of Example 3 was introduced exhibits a further excellent bone regeneration effect.

### EXAMPLE 9

### Evaluation of Apoptosis To evaluate whether apoptosis occurs based on HSV-tk gene expression of the

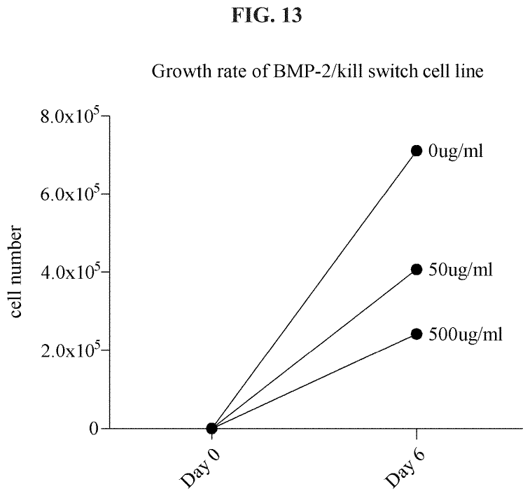

TDF cell line of Example 3, a cell line of Example 3 was treated with gancyclovir in concentrations of 0 μg/ml, 50 μg/ml and 500 μg/ml, respectively, and the number of cells was observed and analyzed for a period of 72 hours to 124 hours, as shown in FIGS. 12 and 13.

Referring to FIGS. 12 and 13, it is confirmed that cell growth is inhibited depending on a ganciclovir treatment concentration and time, which indicates that a kill switch of a cell was activated based on HSV-tk gene expression. Thus, it may be found that the TDF cell line of Example 3 may effectively prevent cells from becoming cancer cells due to self-proliferation, after completing osteogenesis.

### EXAMPLE 10

### Establishment of OB Cell Line

WA01 male embryonic stem cells (WiCell research institute) were dispensed in a 60-phi dish to obtain 10⁶ cells, and then the vector of Example 2 was transfected. After 48 hours had elapsed after transfection, cells that do not contain a neoR gene were killed and cells into which the BMP-2 gene was introduced were selected, through treatment with neomycin for 5 days.

The selected cells were differentiated into an embryoid body (EB) and cultured in an osteogenic differentiation culture solution containing adenosine, to establish a cell line with osteoblasts (OB).

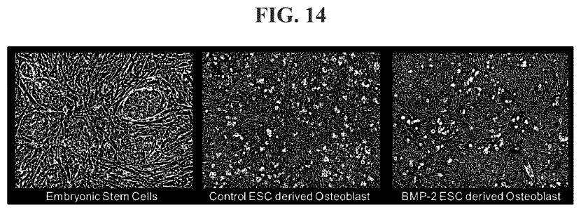

Next, by reacting a loxP gene and Cre protein by transfection of plasmid DNA containing pCAG-Cre into the cell line, BMP-2 and HSV-tk genes were allowed to be expressed. FIG. 14 illustrates images obtained by capturing the ESCs, OBs differentiated by inserting an empty vector or the vector of Example 2.

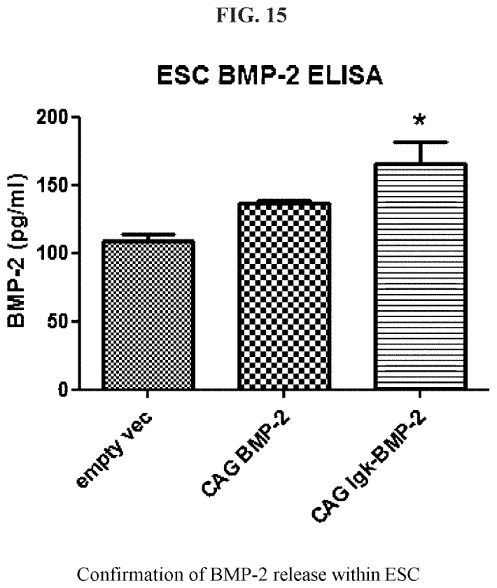

BMP-2 expression of the cell line was confirmed through the ELISA analysis, and the results are shown in FIG. 15. Referring to FIG. 15, it may be confirmed that an OB cell line into which BMP-2 was introduced exhibits enhanced extracellular BMP-2 release in comparison to a control OB cell line into which the empty vector was introduced.

### EXAMPLE 11

### Measurement of Osteogenesis-Related Marker Expression Level

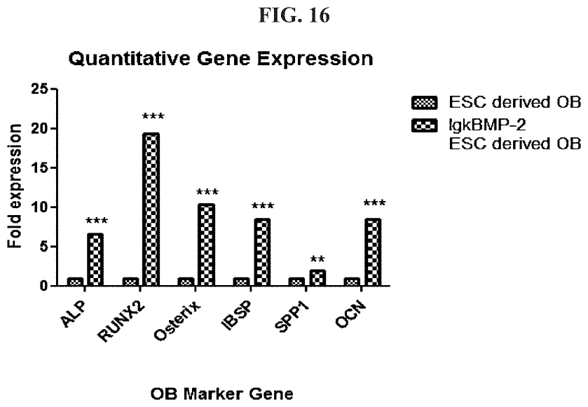

To verify whether the OB cell line of Example 10 increases expression of ALP, RUNX2, OSX, IBSP, SPP1 and OCN that are osteogenesis-related markers, a gene expression level of each marker was measured in the same manner as in Example 6. An empty vector-introduced OB cell line was used as a control group, and the measurement results are shown in FIG. 16.

Referring to FIG. 16, it may be found that the OB cell line of Example 10 increases expression of all the ALP, the RUNX2, the OSX, the IBSP, the SPP1 and the OCN that are osteogenesis markers, which implies that the BMP-2-introduced OB cell line may effectively induce osteogenesis.

### EXAMPLE 12

### Evaluation of Late Osteogenic Induction Ability

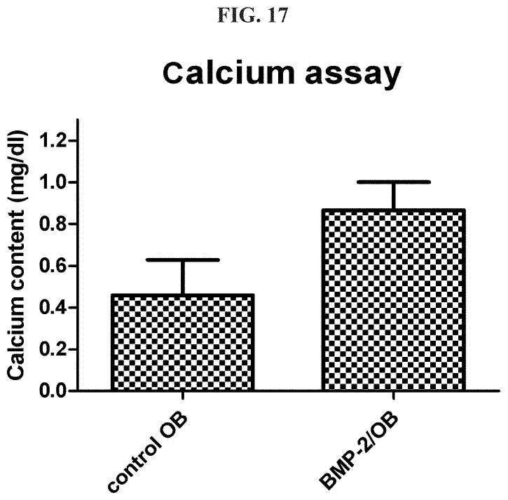

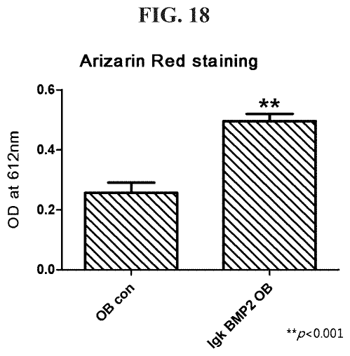

To evaluate a late osteogenic induction ability of the OB cell line of Example 10, a calcium deposition level and a mineral deposition level were measured in the same manner as in Example 7, and the results are shown in FIGS. 17 and 18.

Referring to FIGS. 17 and 18, it may be found that the OB cell line of Example 10 exhibits a calcium deposition level and a mineral deposition level which are about doubled in comparison to a control group in which an empty vector was introduced, which indicates that the late osteogenic induction ability is excellent.

### EXAMPLE 13

### Evaluation of Osteogenesis in Animal Model

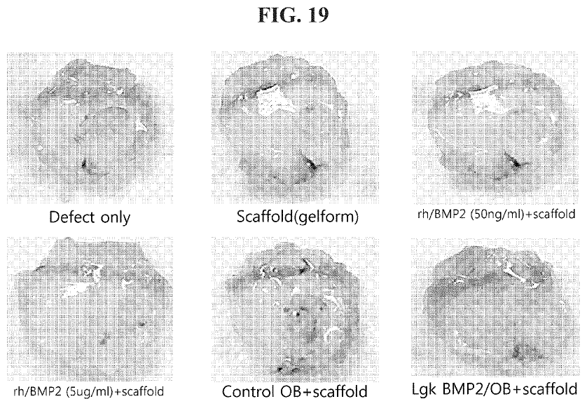

To verify a bone regeneration effect of the OB cell line of Example 10 in a cranial defect animal model, osteogenesis was evaluated using the same H&E tissue staining test method as in Example 8, and the results are shown in FIG. 19.

A case (Defect only) in which no treatment was performed, a case (Scaffold) in which only BCP scaffolds were applied, cases in which BMP-2 injected from the outside was applied together with scaffolds (50 ng/ml and 5 μg/ml), and a case in which an empty vector-introduced OB cell line together with scaffolds were applied were set as control groups.

Referring to FIG. 19, it may be found that when the OB cell line of Example 10 is applied, a more excellent osteogenesis effect may be realized in comparison to the control groups.

### EXAMPLE 14

### Genome Editing of HGPRT Gene—Implementation of Dual Kill Switch

To remove an HGPRT-encoding gene located on an X-chromosome and introduce a dual kill switch system, a Cas9/CRISPR method was performed. An sgRNA (guide RNA) complementary to a PAM site at an exon 8 of an HGPRT gene was cloned. Cas9 plasmid DNA and sgRNA were introduced into the TDF cell line of Example 3 at a ratio of 1:1 using a Neon® transfection method, to create a dual kill switch expression vector.

To verify whether the HGPRT gene is removed, RNA was extracted from a cell line (dual kill switch group) into which the dual kill switch expression vector was introduced and the TDF cell line (control group) of Example 3, the extracted RNA was synthesized into cDNA, and an amount thereof was measured using GAPDH, HGPRT real time Primer. An annealing temperature was 60° C., and the following primers were used:

Forward primer:
TGACACTGGCAAAACAATGCA
Reverse primer:
GGTCCTTTTCACCAGCAAGCT

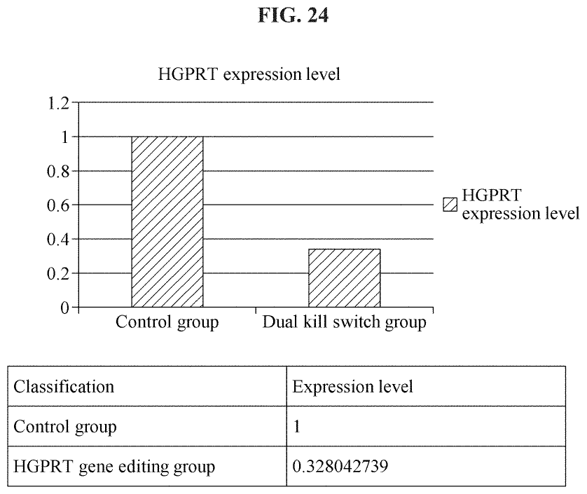

Referring to FIG. 24, it may be confirmed that an HGPRT expression level of the dual kill switch group was measured to be about 0.328 times the control group, and thus most HGPRT genes were removed.

### EXAMPLE 15

### Establishment of Dual Kill Switch Cell Line Using Drug Testing

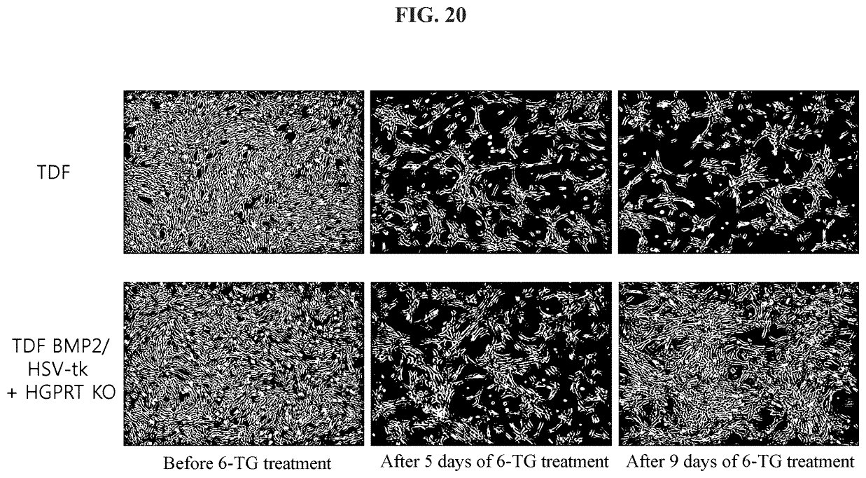

A TDF cell line, knocking out HGPRT genes in Example 14, and the TDF cell line of Example 3 were treated with 6-tioguanine (TG), the number of cells was observed after 5 days and 9 days of the above treatment as shown in FIG. 20. Surviving HGPRT gene knockout cells were isolated.

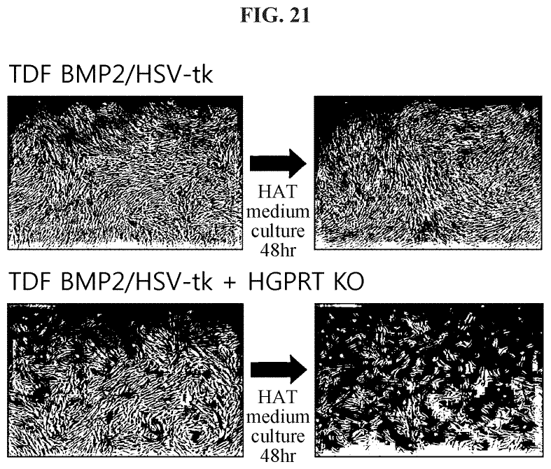

The isolated HGPRT gene knockout cell line and the TDF cell line of Example 3 were cultured in a 6-well plate at a density of 1×10⁴ cells/well. A 50× aminopterins (HAT) stock was diluted in a culture solution and treated with 1× (100 μM hypoxanthine, 0.4 μM aminopterin, 16 μM thymidine). Apoptosis after 48 hours of culturing in the culture solution treated with aminopterin (HAT) was observed and shown in FIG. 21.

Referring to FIG. 20, it may be confirmed that more cells survived in the HGPRT knockout cell line of Example 14 in comparison to the TDF cell line of Example 3 after 9 days of 6-TG treatment. Also, referring to FIG. 21, it may be confirmed that by treatment with aminopterin, the number of cells in the TDF cell line of Example 3 was not almost changed, but that apoptosis was observed in the isolated HGPRT knockout cell line.

While a few example embodiments have been shown and described with reference to the accompanying drawings, it will be apparent to those skilled in the art that various modifications and variations can be made from the foregoing descriptions. For example, adequate effects may be achieved even if the foregoing processes and methods are carried out in different order than described above, and/or the aforementioned elements, such as systems, structures, devices, or circuits are combined or coupled in different forms and modes than as described above or be substituted or switched with other components or equivalents.

Thus, other implementations, alternative example embodiments and equivalents to the claimed subject matter are construed as being within the appended claims.

### Sequence List Free Text

<110> KOREA UNIVERSITY
<120>COMPOSITION FOR PREVENTING OR TREATING BONE DISEASE HAVING EXCELLENT BONE REGENERATION ABILITY
<130> FPC-2018-0109/PCT
<150> KR 10-2017-0020967
<151> 2017-02-16
<150> KR 10-2018-0017137
<151> 2018-02-12
<160> 4
<170> KoPatentIn 3.0
<210> 1
<211> 345
<212> DNA
<213> Homo sapiens

1. A composition for preventing or treating a bone disease, the composition comprising:
a BMP-2-encoding gene;
an HSV-tk-encoding gene; and
a stem cell into which a dual kill switch expression vector in which an HGPRT-encoding gene is knocked out is introduced, or a cell differentiated from the stem cell, as an active ingredient.

2. The composition of claim 1, wherein the stem cell is an embryonic stem cell (ESC) or a mesenchymal stein cell (MSC).

3. The composition of claim 1, wherein the cell differentiated from the stem cell is a fibroblast or an osteoblast.

4. The composition of claim 3, wherein the fibroblast is a teratoma-derived fibroblast (TDF).

5. The composition of claim 1, wherein the bone disease is at least one selected from the group consisting of a bone defect, osteoporosis, an osteoporotic fracture, a diabetic fracture, a nonunion fracture, osteogenesis imperfecta and osteomalacia.

6. The composition of claim 1, wherein the composition increases expression of at least one marker selected from the group consisting of alkaline phosphatase (ALP), integrin binding sialoprotein (IBSP), runt-related transcription factor 2 (RUNX2), osterix (OSX), secreted phosphoprotein 1 (SPP1) and osteocalcin (OCN).

7. The composition of claim 1, further comprising a scaffold.

8. The composition of claim 7, wherein the scaffold comprises polycaprolactone (PCL) or biphasic calcium phosphate (BCP).

9. A method of preparing a composition for preventing or treating a bone disease, the method comprising:
preparing a vector comprising a BMP-2-encoding gene and an HSV-tk-encoding gene;
knocking out an HGPRT-encoding gene in the vector; and
introducing the vector into a stem cell or a cell differentiated from the stem cell.


SEQUENCE LISTING (4 sequences)

SEQ ID NO: 1 - DNA, 345 bp, Homo sapiens
caagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctag

SEQ ID NO: 2 - DNA, 593 bp, Homo sapiens
aattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 3 - DNA, 1131 bp, Homo sapiens
atggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactga

SEQ ID NO: 4 - DNA, 366 bp, Artificial Sequence
  Lgk leading peptide binding BMP-2 gene
gttccaggttccactggtgaccaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctag